(19) United States
(12) Patent Application Publication
(10) Pub. No.: US 2020/0008856 A1
(43) Pub. Date: Jan. 9, 2020

(54) CRYOGENIC BALLOON PRESSURE SENSOR ASSEMBLY

(71) Applicant: Boston Scientific Scimed Inc., Maple Grove, MN (US)

(72) Inventors: Chadi Harmouche, Saint-Laurent (CA); Eric Ryba, San Diego, CA (US)

(21) Appl. No.: 16/576,896

(22) Filed: Sep. 20, 2019

Related U.S. Application Data
(63) Continuation of application No. PCT/US2018/020371, filed on Mar. 1, 2018.
(60) Provisional application No. 62/479,798, filed on Mar. 31, 2017.

(51) Int. Cl. A61B 18/02

(52) U.S. Cl. CPC A61B 18/02; A61B 2018/0022

(57) ABSTRACT

A cryogenic balloon catheter system includes an inflatable balloon, a handle assembly and a pressure sensor. The inflatable balloon has a balloon interior. The pressure sensor senses a balloon pressure within the balloon interior. In various embodiments, the pressure sensor can be positioned within the balloon interior, within the handle assembly and/or between the inflatable balloon and the handle assembly. The cryogenic balloon catheter system also includes a controller that receives a sensor output from the pressure sensor. The controller can control injection of a cooling fluid to the balloon interior and/or removal of the cooling fluid from the balloon interior based upon the sensor output. The cryogenic balloon catheter system can also include an injection proportional valve, an exhaust proportional valve, an injection flow sensor and/or an exhaust flow sensor.

### CROSS-REFERENCE TO RELATED APPLICATION

This application is a continuation of International Application No. PCT/US18/20371, with an international filing date of Mar. 1, 2018, which claims the benefit of U.S. Provisional Application No. 62/479,798, filed on Mar. 31, 2017, and entitled “CRYOGENIC BALLOON PRESSURE SENSOR ASSEMBLY”. As far as permitted, the contents of International Application No. PCT/US18/20371 and U.S. Provisional Application Ser. No. 62/479,798 are incorporated herein by reference.

### TECHNICAL FIELD

The present invention relates to medical devices and methods for cryoablation. More specifically, the invention relates to devices and methods for controlling pressure within a cryoablation balloon catheter.

### BACKGROUND

Cardiac arrhythmias involve an abnormality in the electrical conduction of the heart and are a leading cause of stroke, heart disease, and sudden cardiac death. Treatment options for patients with arrhythmias include medications, implantable devices, and catheter ablation of cardiac tissue.

Catheter ablation involves delivering ablative energy to tissue inside the heart to block aberrant electrical activity from depolarizing heart muscle cells out of synchrony with the heart's normal conduction pattern. This procedure is performed by positioning the tip of a catheter adjacent to diseased or targeted tissue in the heart. The energy delivery component of the system is typically at or near the most distal (furthest from the operator) portion of the catheter, and often at a distal tip of the device. Various forms of energy, such as cryogenic energy as one example, are used to ablate diseased heart tissue. During a cryogenic ablation procedure, with the aid of a guide wire, the distal tip of the catheter is positioned adjacent to diseased tissue, at which time the cryogenic energy can be delivered to create tissue necrosis, rendering the ablated tissue incapable of conducting electrical signals.

Atrial fibrillation (AF) is one of the most common arrhythmias treated using catheter ablation. In the earliest stages of the disease, paroxysmal AF, the treatment strategy involves isolating the pulmonary veins from the left atrial chamber. Recently, the use of techniques known as “balloon cryotherapy” catheter procedures to treat AF have increased. During therapy, a balloon is placed inside or against the ostium of a pulmonary vein to occlude the pulmonary vein. Pulmonary vein occlusion is typically a strong indicator that complete circumferential contact is achieved between the balloon and pulmonary vein for optimal heat transfer during ablation. Some advantages of balloon cryotherapy include ease of use, shorter procedure times and improved patient outcomes.

In balloon ablation procedures, such as cryoablations, full balloon contact with the surface of the tissue is critical for successful clinical outcome. For pulmonary vein ablations for example, the physician needs to occlude the veins with the balloon to reduce or eliminate blood flow around the ablation area and increase balloon to tissue contact to achieve better ablation results. One way this is accomplished is by inflating the balloon through either a fixed volume of cooling fluid or a very low cooling fluid flow in which there in no significant cooling occurring. The physician can then push the balloon against the ostium and assess occlusion quality. Once sufficient occlusion is confirmed, ablation can be initiated where the balloon goes from a no cooling inflated state to a cooling inflated state. This can be achieved through a combination of increasing the cooling fluid injection pressure and controlling the return back pressure of the resultant cooling fluid gas to maintain the balloon pressure above the surrounding pressure in order to maintain proper inflation of the balloon during various phases of the cryoablation procedure. One of the main control parameters required to achieve this process is knowing and/or monitoring the pressure value inside the balloon.

One conventional method that is being used is inhibiting the balloon from deflating between the inflation phase and the ablation by estimating the balloon pressure through one or more sensors located in a console as a signal to control the return back pressure. This method is not altogether satisfactory. One distinct disadvantage of sensing pressure at a distant location is that it is very difficult to correlate the pressure at the distant location to the actual balloon pressure. Pressures at any given location will change as a function of flowrate and/or thermal effects.

Additionally, due to the very nature of the system fluid flow, there will be time delays between pressures and/or changes in pressure at one location versus another location. Relatively small pressure changes within the balloon of only a couple pounds per square inch (psi) can cause the balloon to either collapse due to the pressure being too low, or create a higher than desired pressure that may affect patient safety. With this conventional methodology, the lack of having accurate and/or direct pressure balloon measurement can cause the balloon pressure to fluctuate between inflation and ablation leading to change in balloon stiffness and size. This can cause the balloon to “pop out” of the veins and lose proper occlusion. Another effect of the change in balloon pressure can lead to tissue damage such as vein stenosis if the balloon is too far in the vein during inflation. The increase in balloon pressure can force the balloon against the pulmonary vein walls potentially leading to tissue damage.

### SUMMARY

The present invention is directed toward a cryogenic balloon catheter system for treating a condition in a patient. In one embodiment, the cryogenic balloon catheter system includes an inflatable balloon and a pressure sensor. The inflatable balloon is positioned within the body and has a balloon interior. The pressure sensor senses a balloon pressure within the balloon interior. In one embodiment, the pressure sensor is positioned within the balloon interior.

In certain embodiments, the cryogenic balloon catheter system also includes a controller that receives a sensor output from the pressure sensor. The controller can control injection of a cooling fluid to the balloon interior based at least in part upon the sensor output. Additionally, or in the alternative, the controller can control removal of the cooling fluid from the balloon interior based at least in part upon the sensor output.

In various embodiments, the cryogenic balloon catheter system can also include an injection proportional valve. In some such embodiments, the controller can control the injection proportional valve based at least partially upon the sensor output.

In some embodiments, the cryogenic balloon catheter system can also include an exhaust proportional valve. In some such embodiments, the controller can control the exhaust proportional valve based at least partially upon the sensor output.

In certain embodiments, the cryogenic balloon catheter system can also include an injection flow sensor that senses a flow of the cooling fluid to the balloon interior. In some such embodiments, the controller receives information from the injection flow sensor, and the controller controls injection of the cooling fluid to the balloon interior based at least in part upon the information from the injection flow sensor.

In various embodiments, the cryogenic balloon catheter system can also include an exhaust flow sensor that senses a flow of the cooling fluid from the balloon interior. In some such embodiments, the controller can receive information from the exhaust flow sensor, and can control removal of the cooling fluid from the balloon interior based at least in part upon the information from the exhaust flow sensor.

In another embodiment, the cryogenic balloon catheter system includes an inflatable balloon, a handle assembly and a pressure sensor. The inflatable balloon is positioned within the body and has a balloon interior. The pressure sensor senses a balloon pressure within the balloon interior. The handle assembly is coupled to the inflatable balloon, and is configured to be positioned outside the body. In one embodiment, the pressure sensor is positioned within the handle assembly.

In yet another embodiment, the cryogenic balloon catheter system includes an inflatable balloon, a handle assembly and a pressure sensor. The inflatable balloon has a balloon interior. The pressure sensor senses a balloon pressure within the balloon interior. The handle assembly is coupled to the inflatable balloon, and is configured to be positioned outside the body. In this embodiment, the pressure sensor is positioned between the handle assembly and the balloon interior.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic view illustration of a patient and one embodiment of a cryogenic balloon catheter system including a cryogenic balloon pressure sensor assembly having features of the present invention;

FIG. 2 is a simplified side view of a portion of the patient and a portion of an embodiment of the cryogenic balloon catheter system including one embodiment of the cryogenic balloon pressure sensor assembly;

FIG. 3 is a simplified side view of a portion of the patient and a portion of an embodiment of the cryogenic balloon catheter system including another embodiment of the cryogenic balloon pressure sensor assembly;

FIG. 4 is a simplified schematic diagram illustrating an embodiment of the cryogenic balloon catheter system including one embodiment of the cryogenic balloon pressure sensor assembly; and

FIG. 5 is a simplified schematic diagram illustrating an embodiment of the cryogenic balloon catheter system including another embodiment of the cryogenic balloon pressure sensor assembly.

### DETAILED DESCRIPTION

Embodiments of the present invention are described herein in the context of a cryogenic balloon catheter system (also sometimes referred to herein as a “catheter assembly”) which includes a cryogenic balloon pressure sensor assembly (also sometimes referred to herein as a “pressure sensor assembly”). Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

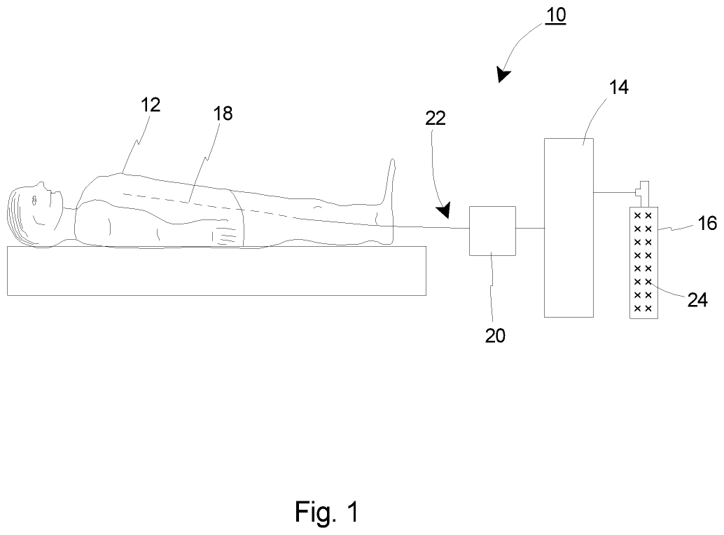

FIG. 1 is a schematic side view illustration of one embodiment of a medical device 10 for use with a patient 12, which can be a human being or an animal. Although the specific medical device 10 shown and described herein pertains to and refers to a cryogenic balloon catheter system 10, it is understood and appreciated that other types of medical devices 10 can equally benefit by the teachings provided herein. The design of the cryogenic balloon catheter system 10 can be varied. In certain embodiments such as the embodiment illustrated in FIG. 1, the cryogenic balloon catheter system 10 can include one or more of a control system 14, a fluid source 16, a balloon catheter 18, a handle assembly 20, a control console 22, a graphical display 24 and a pressure sensor assembly 25. It is understood that although FIG. 1 illustrates the structures of the cryogenic balloon catheter system 10 in a particular position, sequence and/or order, these structures can alternatively be located in any suitable position, sequence and/or order different than that illustrated in FIG. 1.

In various embodiments, the control system 14 can control release and/or retrieval of a cryogenic fluid 26 to and/or from the balloon catheter 18. In various embodiments, the control system 14 can control activation and/or deactivation of one or more other processes of the balloon catheter 18. Additionally, or in the alternative, the control system 14 can receive electrical signals, including data and/or other information (hereinafter sometimes referred to as “sensor output”) from various structures within the cryogenic balloon catheter system 10. In some embodiments, the control system 14 can assimilate and/or integrate the sensor output, and/or any other data or information received from any structure within the cryogenic balloon catheter system 10. Additionally, or in the alternative, the control system 14 can control positioning of portions of the balloon catheter 18 within the body of the patient 12, and/or can control any other suitable functions of the balloon catheter 18.

The fluid source 16 contains the cryogenic fluid 26, which is delivered to the balloon catheter 18 with or without input from the control system 14 during a cryoablation procedure. The type of cryogenic fluid 26 that is used during the cryoablation procedure can vary. In one non-exclusive embodiment, the cryogenic fluid 26 can include liquid nitrous oxide. However, any other suitable cryogenic fluid 26 can be used.

The balloon catheter 18 is inserted into the body of the patient 12. In one embodiment, the balloon catheter 18 can be positioned within the body of the patient 12 using the control system 14. Alternatively, the balloon catheter 18 can be manually positioned within the body of the patient 12 by a health care professional (also sometimes referred to herein as an “operator”). In certain embodiments, the balloon catheter 18 is positioned within the body of the patient 12 utilizing the sensor output from the balloon catheter 18. In various embodiments, the sensor output is received by the control system 14, which then can provide the operator with information regarding the positioning of the balloon catheter 18. Based at least partially on the sensor output feedback received by the control system 14, the operator can adjust the positioning of the balloon catheter 18 within the body of the patient 12. While specific reference is made herein to the balloon catheter 18, it is understood that any suitable type of medical device and/or catheter may be used.

The handle assembly 20 is handled and used by the operator to operate, position and control the balloon catheter 18. The design and specific features of the handle assembly 20 can vary to suit the design requirements of the cryogenic balloon catheter system 10. In the embodiment illustrated in FIG. 1, the handle assembly 20 is separate from, but in electrical and/or fluid communication with the control system 14, the fluid source 16 and/or the graphical display 24. In some embodiments, the handle assembly 20 can integrate and/or include at least a portion of the control system 14 within an interior of the handle assembly 20. It is understood that the handle assembly 20 can include fewer or additional components than those specifically illustrated and described herein.

In the embodiment illustrated in FIG. 1, the control console 22 includes the control system 14, the fluid source 16 and the graphical display 24. However, in alternative embodiments, the control console 22 can contain additional structures not shown or described herein. Still alternatively, the control console 22 may not include various structures that are illustrated within the control console 22 in FIG. 1. For example, in one embodiment, the control console 22 does not include the graphical display 24.

The graphical display 24 provides the operator of the cryogenic balloon catheter system 10 with information that can be used before, during and after the cryoablation procedure. The specifics of the graphical display 24 can vary depending upon the design requirements of the cryogenic balloon catheter system 10, or the specific needs, specifications and/or desires of the operator.

In one embodiment, the graphical display 24 can provide static visual data and/or information to the operator. In addition, or in the alternative, the graphical display 24 can provide dynamic visual data and/or information to the operator, such as video data or any other data that changes over time. Further, in various embodiments, the graphical display 24 can include one or more colors, different sizes, varying brightness, etc., that may act as alerts to the operator. Additionally, or in the alternative, the graphical display can provide audio data or information to the operator.

As an overview, and as provided in greater detail herein, the pressure sensor assembly 25 can sense and/or monitor a balloon pressure within a portion of the balloon catheter 18. Further, the pressure sensor assembly 25 can provide pressure data and/or information to other structures, within the cryogenic balloon catheter system 10, e.g., the control system 14, which can be used to control various functions of the cryogenic balloon catheter system 10 as described herein.

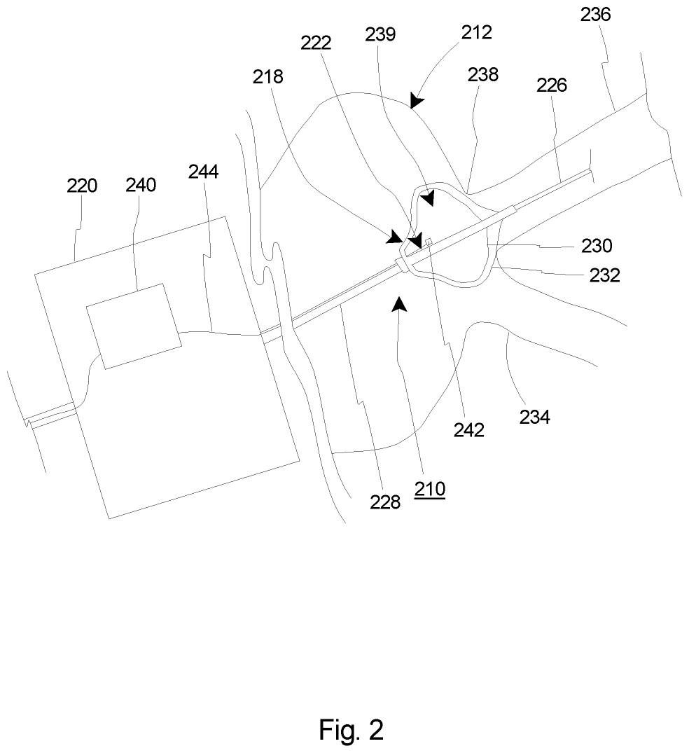

FIG. 2 is a simplified side view of a portion of one embodiment of the cryogenic balloon catheter system 210 and a portion of a patient 212. The control system 14 (illustrated in FIG. 1) and the cooling fluid source 16 (illustrated in FIG. 1) have been omitted from FIG. 2 for clarity. In the embodiment illustrated in FIG. 2, the cryogenic balloon catheter system 210 includes a balloon catheter 218, a handle assembly 220 and a pressure sensor assembly 225.

The design of the balloon catheter 218 can be varied to suit the design requirements of the cryogenic balloon catheter system 210. In this embodiment, the balloon catheter 218 includes one or more of a guidewire 227, a catheter shaft 228, an inner inflatable balloon 230 (sometimes referred to herein simply as an “inflatable balloon”) and an outer inflatable balloon 232. It is understood that the balloon catheter 218 can include other structures as well. However, for the sake of clarity, these other structures have been omitted from the Figures. In the embodiment illustrated in FIG. 2, the balloon catheter 218 is positioned within the circulatory system 234 of the patient 212. The guidewire 227 is inserted into a pulmonary vein 236 of the patient 212, and the catheter shaft 228 and the balloons 230, 232 are moved along the guidewire 227 to near an ostium 238 of the pulmonary vein 236.

In one embodiment, the inner inflatable balloon 230 can be made from a relatively non-compliant or semi-compliant material. Some representative materials suitable for this application include PET (polyethylene terephthalate), nylon, polyurethane, and co-polymers of these materials such as polyether block amide (PEBA), known under its trade name as PEBAX® (supplier Arkema), as nonexclusive examples. In another embodiment, a polyester block copolymer known in the trade as Hytrel® (DuPont™) is also a suitable material for the inner inflatable balloon 230. The inner inflatable balloon 230 can be notable in that it can be relatively inelastic to the relatively more compliant outer inflatable balloon 232. The inner inflatable balloon 230 defines an inner balloon interior 239 (also sometimes referred to herein simply as an “balloon interior”).

In one embodiment, the outer inflatable balloon 232 can be made from a relatively compliant material. Such materials are well known in the art. One nonexclusive example is aliphatic polyether polyurethanes which carbon atoms are linked in open chains, including paraffins, olefins, and acetylenes. Another available example goes by the trade name Tecoflex® (Lubrizol). Other available polymers from the polyurethane class of thermoplastic polymers with exceptional elongation characteristics are also suitable for use as the outer inflatable balloon 232.

During use, the inner inflatable balloon 230 can be partially or fully inflated so that at least a portion of the inner inflatable balloon 230 expands against a portion of the outer inflatable balloon 232 (although a space is shown between the inner inflatable balloon 230 and the outer inflatable balloon 232 in FIG. 2 for clarity). As provided herein, once the inner inflatable balloon 230 is sufficiently inflated, the outer inflatable balloon 232 can then be positioned within the circulatory system 234 of the patient 212 to abut and/or form a seal with the ostium 238 of the pulmonary vein 236 to be treated.

The design of the handle assembly 220 can vary. In the embodiment illustrated in FIG. 2, the handle assembly 220 can include circuitry 240 that can form a portion of the control system 14. Alternatively, the circuitry 240 can transmit electrical signals such as the sensor output or otherwise provide data to the control system 14 as described herein. Additionally, or in the alternative, the circuitry 240 can receive electrical signals or data from the pressure sensor assembly 225. In one embodiment, the circuitry 240 can include a printed circuit board having one or more integrated circuits, or any other suitable circuitry. In an alternative embodiment, the circuitry 240 can be omitted, or can be included within the control system 14, which in various embodiments can be positioned outside of the handle assembly 220.

The pressure sensor assembly 225 senses and/or monitors a balloon pressure inside the inner inflatable balloon 230. As used herein, the “balloon pressure” means the pressure inside of the inner inflatable balloon 230 at or substantially contemporaneously with the time the pressure in the inner balloon interior 239 is measured. In the embodiment illustrated in FIG. 2, the pressure sensor assembly 225 can transmit electrical signals to the circuitry 240, which are then processed and sent to the control system 14. In an alternative embodiment, the pressure sensor assembly 225 can transmit electrical signals directly to the control system 14. The design of the pressure sensor assembly 225 can be varied. In the embodiment illustrated in FIG. 2, the pressure sensor assembly 225 includes a pressure sensor 242 and a transmission line 244.

In this embodiment, the pressure sensor 242 is positioned in the inner balloon interior 239. With this design, the pressure sensor 242 can directly sense, measure and/or monitor the balloon pressure within the inner inflatable balloon 230. The pressure sensor 242 sends a sensor output, e.g., electrical signals regarding the balloon pressure, to the circuitry 240 and/or the control system 14 via the transmission line 244. As described in greater detail herein, the control system 14 can then adjust the balloon pressure based at least in part on the information/data provided by the pressure sensor 242.

The specific type of pressure sensor 242 included in the pressure sensor assembly 225 can vary. For example, in one embodiment, the pressure sensor 242 can include a “MEMS” sensor or an optical pressure detector, as nonexclusive examples. Alternatively, another suitable type of pressure sensor 242 can be used.

In certain embodiments, the control system 14 (illustrated in FIG. 1) is configured to process and integrate the sensor output to determine and/or adjust for proper functioning of the cryogenic balloon catheter system 210. Based at least in part on the sensor output, the control system 14 can determine that certain modifications to the functioning of the cryogenic balloon catheter system 210 are required.

The control system 14 can abort the delivery of cryogenic fluid, can increase the fluid flow rate to get more cooling, reduce the fluid flow rate, it can have an initial flow rate to reduce temperature to a set point then change the flow rate to maintain a set temperature. It can change the cycle time or amount of fluid delivery to and from the inner inflatable balloon 230.

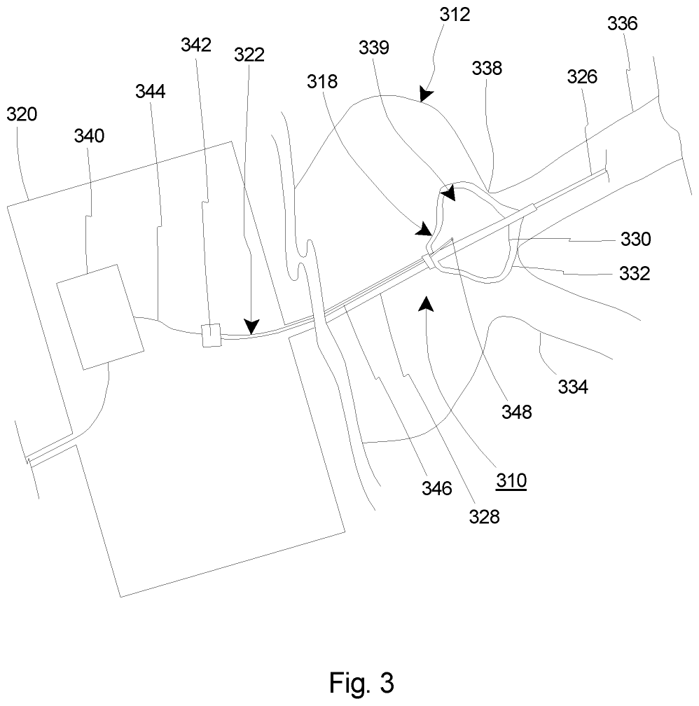

FIG. 3 is a simplified side view of a portion of another embodiment of the cryogenic balloon catheter system 310 and a portion of a patient 312. The control system 14 (illustrated in FIG. 1) and the cooling fluid source 16 (illustrated in FIG. 1) have been omitted from FIG. 3 for clarity. In the embodiment illustrated in FIG. 3, the cryogenic balloon catheter system 310 includes a balloon catheter 318, a handle assembly 320 and a pressure sensor assembly 325.

The design of the balloon catheter 318 can be varied to suit the design requirements of the cryogenic balloon catheter system 310. In this embodiment, the balloon catheter 318 includes one or more of a guidewire 327, a catheter shaft 328, an inner inflatable balloon 330 and an outer inflatable balloon 332. It is understood that the balloon catheter 318 can include other structures as well. However, for the sake of clarity, these other structures have been omitted from the Figures. In the embodiment illustrated in FIG. 3, the balloon catheter 318 is positioned within the circulatory system 334 of the patient 312. The guidewire 327 is inserted into a pulmonary vein 336 of the patient 312, and the catheter shaft 328 and the balloons 330, 332 are moved along the guidewire 327 to near an ostium 338 of the pulmonary vein 336.

In the embodiment illustrated in FIG. 3, the inner inflatable balloon 330 and the outer inflatable balloon 332 are substantially similar to those previously described herein. Further, the functioning of the inner inflatable balloon 330 and the outer inflatable balloon 332 is substantially similar to that previously described herein. The inner inflatable balloon 330 defines an inner balloon interior 339.

The design of the handle assembly 320 can vary. In the embodiment illustrated in FIG. 3, the handle assembly 320 can include circuitry 340 that can form a portion of the control system 14. In this embodiment, the circuitry 340 can function substantially similarly to the circuitry previously described herein. In an alternative embodiment, the circuitry 340 can be omitted, or the circuitry 340 can be included within the control system 14, which in various embodiments can be positioned outside of the handle assembly 320.

The pressure sensor assembly 325 senses and/or monitors a balloon pressure inside the inner inflatable balloon 330. As used herein, the “balloon pressure” means the pressure inside of the inner inflatable balloon 330 at or substantially contemporaneously with the time the pressure in the inner balloon interior 339 is measured. In the embodiment illustrated in FIG. 3, the pressure sensor assembly 325 can transmit electrical signals, e.g. sensor output, to the circuitry 340, which are then processed and sent to the control system 14. In an alternative embodiment, the pressure sensor assembly 325 can transmit electrical signals directly to the control system 14. The design of the pressure sensor assembly 325 can be varied. In the embodiment illustrated in FIG. 3, the pressure sensor assembly 325 includes a pressure sensor 342, a transmission line 344 and a tubular member 346 that defines a sensor lumen 348 (an interior of the tubular member 346).

In certain embodiments, the pressure sensor 342 is positioned outside of the inner balloon interior 339. For example, in the embodiment illustrated in FIG. 3, the pressure sensor 342 is positioned within the handle assembly 320. Alternatively, the pressure sensor 342 can be positioned anywhere between the inner inflatable balloon 330 and the handle assembly 320. Still alternatively, the pressure sensor 342 can be positioned between the handle assembly 320 and the control system 14.

In the embodiment illustrated in FIG. 3, the tubular member 346 extends from the pressure sensor 342 to the inner balloon interior 339. The pressure sensor 342 is in fluid communication with the inner balloon interior 339 via the tubular member 346. The tubular member 346 can be a relatively small diameter tube that can transmit the balloon pressure within the inner balloon interior 339 directly to the pressure sensor 342. The pressure sensor 342 then sends a sensor output, e.g., electrical signals regarding the balloon pressure, to the circuitry 340 and/or the control system 14 via the transmission line 344. As provided herein, the control system 14 can then adjust the balloon pressure based at least in part on the information/data provided by the pressure sensor 342.

The specific type of pressure sensor 342 included in the pressure sensor assembly 325 can vary. For example, in one embodiment, the pressure sensor 342 can include a “MEMS” sensor or an optical pressure detector, as nonexclusive examples. Alternatively, another suitable type of pressure sensor 342 can be used.

In certain embodiments, the control system 14 (illustrated in FIG. 1) is configured to process and integrate the sensor output to determine and/or adjust for proper functioning of the cryogenic balloon catheter system 310. Based at least in part on the sensor output, the control system 14 can determine that certain modifications to the functioning of the cryogenic balloon catheter system 310 are required.

The control system 14 can abort the delivery of cryogenic fluid, can increase the fluid flow rate to get more cooling, reduce the fluid flow rate, it can have an initial flow rate to reduce temperature to a set point then change the flow rate to maintain a set temperature. It can change the cycle time or amount of fluid delivery to and from the inner inflatable balloon 330.

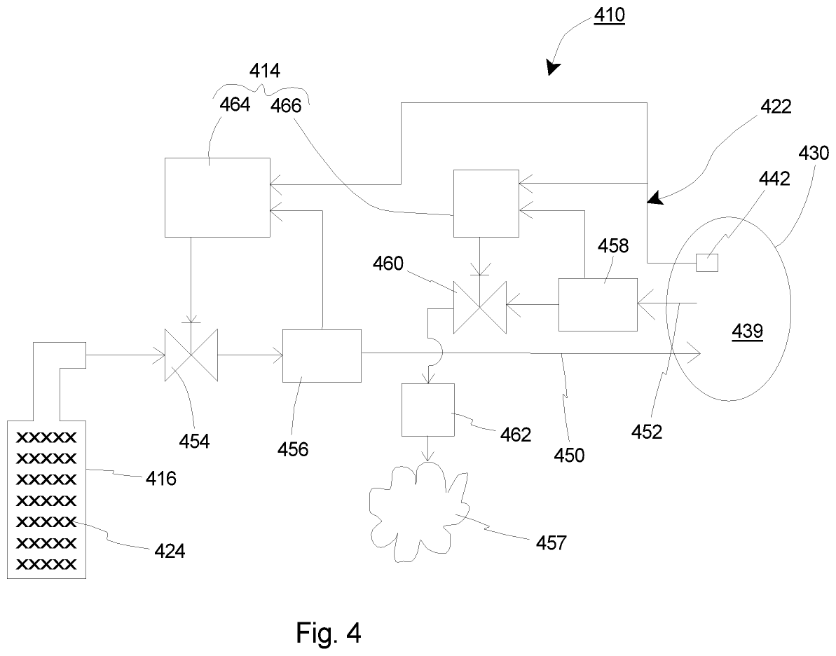

FIG. 4 is a simplified schematic diagram illustrating one embodiment of the cryogenic balloon catheter system 410. In this embodiment, the cryogenic balloon catheter system 410 includes a control system 414, a fluid source 416 containing a cooling fluid 426, a pressure sensor assembly 425, an inner inflatable balloon 430 having an inner balloon interior 439, an injection line 450, and an exhaust line 452. In this embodiment, the cryogenic balloon pressure sensor assembly 425 can function substantially similar to that previously described with respect to FIG. 2. More specifically, in this embodiment, the pressure sensor 442 is positioned within the inner balloon interior 439.

In this embodiment, the injection line 450 receives the cooling fluid 426 in a liquid state from the fluid source 416 and delivers the cooling fluid 426 to the inner balloon interior 439. The injection line 450 can vary. In the embodiment illustrated in FIG. 4, the injection line 450 can include one or more of an injection proportional valve 454 and/or an injection flow sensor 456. The injection proportional valve 454 can regulate the flow and/or pressure of the cooling fluid 426 to the inner balloon interior 439. The injection flow sensor 456 can sense and/or monitor a flow rate of the cooling fluid 426 during an injection process.

Further, in this embodiment, the exhaust line 452 receives the cooling fluid 426 in a gaseous state from the inner balloon interior 439 and delivers the cooling fluid 426 as exhaust 457 to a suitable location outside of the patient 12 (illustrated in FIG. 1). The exhaust line 452 can vary. In the embodiment illustrated in FIG. 4, the exhaust line 452 can include one or more of an exhaust flow sensor 458, an exhaust proportional valve 460 and/or a vacuum pump 462. The exhaust flow sensor 458 can sense and/or monitor a flow rate of the cooling fluid 426 during removal of the cooling fluid 426 from the inner balloon interior 439. The exhaust proportional valve 460 can regulate the flow and/or pressure of the cooling fluid 426 from the inner balloon interior 439.

In the embodiment illustrated in FIG. 4, the control system 414 can include an injection line controller 464 and/or an exhaust line controller 466. In this embodiment, the injection line controller 464 and/or the exhaust line controller 466 can receive the sensor output from the pressure sensor assembly 425. Further, in certain embodiments, the injection line controller 464 can receive injection flow sensor information from the injection flow sensor 456, and the exhaust line controller 466 can receive exhaust flow sensor information from the exhaust flow sensor 458. In one embodiment, one or both of the controllers 464, 466, can include a control loop feedback mechanism such as a proportional-integral-derivative controller (PID controller).

With this design, based on the balloon pressure and/or the flow rates, the injection line controller 464 can better control the injection of cooling fluid 426 to the inner balloon interior 439, and the exhaust line controller 466 can better control the removal and exhaust of the cooling fluid 426 from the inner balloon interior 439 and out of the patient 12.

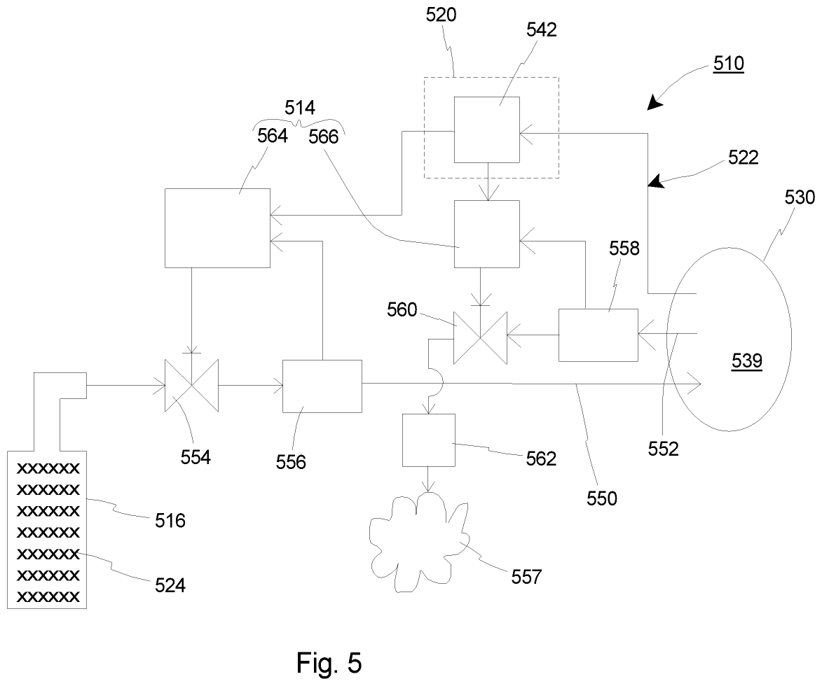

FIG. 5 is a simplified schematic diagram illustrating one embodiment of the cryogenic balloon catheter system 510. In this embodiment, the cryogenic balloon catheter system 510 includes a control system 514, a fluid source 516 containing a cooling fluid 526, a pressure sensor assembly 525, an inner inflatable balloon 530 having an inner balloon interior 539, an injection line 550, and an exhaust line 552. In this embodiment, the cryogenic balloon pressure sensor assembly 525 can function substantially similar to that previously described with respect to FIG. 3. More specifically, in this embodiment, the pressure sensor 542 is positioned outside of the inner balloon interior 539. More specifically, in one such embodiment, the pressure sensor 542 is positioned within the handle assembly 520. However, it is recognized that the pressure sensor 542 can equally be positioned between the inner balloon interior 539 and the handle assembly 520, or between the handle assembly 520 and the controller 514.

In this embodiment, the injection line 550 receives the cooling fluid 526 in a liquid state from the fluid source 516 and delivers the cooling fluid 526 to the inner balloon interior 539. The injection line 550 can vary. In the embodiment illustrated in FIG. 5, the injection line 550 can include one or more of an injection proportional valve 554 and/or an injection flow sensor 556. The injection proportional valve 554 can regulate the flow of the cooling fluid 526 to the inner balloon interior 539. The injection flow sensor 556 can sense and/or monitor a flow rate of the cooling fluid 526 during an injection process.

Further, in this embodiment, the exhaust line 552 receives the cooling fluid 526 in a gaseous state from the inner balloon interior 539 and delivers the cooling fluid 526 as exhaust 557 to a suitable location outside of the patient 12 (illustrated in FIG. 1). The exhaust line 552 can vary. In the embodiment illustrated in FIG. 5, the exhaust line 552 can include one or more of an exhaust flow sensor 558, an exhaust proportional valve 560 and/or a vacuum pump 562. The exhaust flow sensor 558 can sense and/or monitor a flow rate of the cooling fluid 526 during removal of the cooling fluid 526 from the inner balloon interior 539. The exhaust proportional valve 560 can regulate the flow of the cooling fluid 526 from the inner balloon interior 539.

In the embodiment illustrated in FIG. 5, the control system 514 can include an injection line controller 564 and/or an exhaust line controller 566. In this embodiment, the injection line controller 564 and/or the exhaust line controller 566 can receive the sensor output from the pressure sensor assembly 525. Further, in certain embodiments, the injection line controller 564 can receive injection flow sensor information from the injection flow sensor 556, and the exhaust line controller 566 can receive exhaust flow sensor information from the exhaust flow sensor 558. In one embodiment, one or both of the controllers 564, 566, can include a control loop feedback mechanism such as a proportional-integral-derivative controller (PID controller).

With this design, based on the balloon pressure and/or the flow rates, the injection line controller 564 can better control the injection of cooling fluid 526 to the inner balloon interior 539, and the exhaust line controller 566 can better control the pressure during removal of the cooling fluid 526 from the inner balloon interior 539 and out of the patient 12.

It is understood that although a number of different embodiments of the cryogenic balloon catheter system 10 have been illustrated and described herein, one or more features of any one embodiment can be combined with one or more features of one or more of the other embodiments, provided that such combination satisfies the intent of the present invention.

While a number of exemplary aspects and embodiments of a cryogenic balloon catheter system 10 have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

We claim:

1. A cryoballoon catheter for use in a cryogenic balloon catheter system for treating a condition in a patient, the cryoballoon catheter comprising:
a flexible catheter shaft having a proximal end portion and a distal end portion;
an inflatable balloon carried by the distal end portion of the shaft and configured to be positioned proximate a treatment site of the patient, the inflatable balloon having a balloon interior;
a handle assembly that is coupled to the proximal end of the catheter shaft and configured to be positioned external to the patient; and
a pressure sensor positioned within the handle assembly, the pressure sensor configured to sense a balloon pressure within the balloon interior.

2. The cryoballoon catheter of claim 1, further comprising a tubular member that allows fluid communication between the balloon interior and the pressure sensor.

3. The cryoballoon catheter of claim 2, wherein the tubular member is disposed within the catheter shaft and extends to within the balloon interior.

4. The cryoballoon catheter of claim 3, wherein the tubular member defines a sensor lumen configured to transmit the balloon pressure from the balloon interior to the pressure sensor.

5. The cryoballoon catheter of claim 4, wherein the inflatable balloon includes an inner inflatable balloon disposed within an outer inflatable balloon, and wherein the balloon interior is defined by the inner inflatable balloon.

6. The cryoballoon catheter of claim 5, further comprising sensor circuitry disposed within the handle and electrically coupled to the pressure sensor.

7. The cryoballoon catheter of claim 6, wherein the sensor circuitry is configured to be electrically coupled to a controller disposed external to the cryoballoon catheter.

8. A cryogenic balloon catheter system for treating a condition in a patient, the cryogenic balloon catheter system comprising:
a cryoballoon catheter comprising:
a flexible catheter shaft having a proximal end portion and a distal end portion;
an inflatable balloon carried by the distal end portion of the shaft and configured to be positioned proximate a treatment site of the patient, the inflatable balloon having a balloon interior;
a handle assembly that is coupled to the proximal end of the catheter shaft and configured to be positioned external to the patient; and
a pressure sensor positioned within the handle assembly, the pressure sensor configured to sense a balloon pressure within the balloon interior;
a fluid source containing a cryogenic fluid;
an injection line fluidly coupled to the fluid source and the balloon interior;
an exhaust line fluidly coupled to the balloon interior; and
a control system configured to selectively control an injection of the cryogenic fluid to the balloon interior through the injection line, and to selectively control removal of the cryogenic fluid from the balloon interior through the exhaust line.

9. The cryogenic balloon catheter system of claim 8, wherein the control system includes an injection controller operatively coupled to the pressure sensor and configured to receive a sensor output from the pressure sensor and to control injection of the cryogenic fluid to the balloon interior based at least in part upon the sensor output.

10. The cryogenic balloon catheter system of claim 9, further comprising an injection proportional valve disposed in the injection line, the injection controller configured to control the injection proportional valve based at least in part upon the sensor output.

11. The cryogenic balloon catheter system of claim 10, wherein the control system further comprises an exhaust controller configured to control removal of the cryogenic fluid from the balloon interior through the exhaust line based at least in part upon the sensor output.

12. The cryogenic balloon catheter system of claim 11, further comprising an exhaust proportional valve disposed in the exhaust line, the exhaust controller configured to control the exhaust proportional valve based at least in part upon the sensor output.

13. The cryogenic balloon catheter system of claim 12, further comprising an injection flow sensor configured to sense an injection flow of the cryogenic fluid to the balloon interior.

14. The cryogenic balloon catheter system of claim 13 further comprising an exhaust flow sensor configured to sense an exhaust flow of the cooling fluid from the balloon interior.

15. The cryogenic balloon catheter system of claim 14, wherein the control system is further configured to receive information from the injection flow sensor and the exhaust flow sensor.

16. A method of controlling pressure within an inflatable balloon of a cryoballoon catheter having a catheter shaft and a handle assembly, the method comprising:
transmitting balloon pressure within a balloon interior of the inflatable balloon to a pressure sensor disposed within the handle assembly;
generating a pressure sensor output responsive to the balloon pressure transmitted to the pressure sensor; and
controlling one or both of a cryogenic fluid injection flow to the balloon interior and a cryogenic fluid removal flow from the balloon interior based on the pressure sensor output.

17. The method of claim 16, further comprising receiving, by an injection controller, the pressure sensor output, and selectively controlling an injection of the cryogenic fluid to the balloon interior by the injection controller.

18. The method of claim 17, wherein selectively controlling the injection of the cryogenic fluid includes controlling, by the injection controller, an injection proportional valve fluidly coupled to the balloon interior and a cryogenic fluid source.

19. The method of claim 18, further comprising receiving, by an exhaust controller, the pressure sensor output, and selectively controlling removal of the cryogenic fluid from the balloon interior by the exhaust controller.

20. The method of claim 18, wherein selectively controlling the removal of the cryogenic fluid includes controlling, by the exhaust controller, an exhaust proportional valve fluidly coupled to the balloon interior.
